(19) United States
(12) Patent Application Publication
(10) Pub. No.: US 2011/0165019 A1
(43) Pub. Date: Jul. 7, 2011

(54) STERILIZATION OF LIQUID DISPENSING APPARATUS

(75) Inventor: Adam Llywellyn Green, Gloucestershire (GB)

(73) Assignee: WATER WERKZ LIMITED, Gloucestershire (GB)

(21) Appl. No.: 13/056,412

(22) PCT Filed: Jul. 16, 2009
(86) PCT No.: PCT/GB2009/001761

§ 371 Date: Mar. 14, 2011

(30) Foreign Application Priority Data
Jul. 30, 2008 (GB) 0813951.1

(51) Int. Cl. B01J 19/08; A61L 2/02

(52) U.S. Cl. 422/22; 422/186.07; 422/186.2

(57) ABSTRACT

An apparatus and method for periodically sterilizing liquid dispensing apparatus, particularly vending machines, comprises circulating liquid feedstock via a return duct which incorporates an ozone generator. Ozone uptake is enhanced by chilling the circulating liquid. At the end of the sterilizing cycle a carbon based filter may be used to remove ozone from the circulating liquid.

This invention relates to an apparatus and method for sterilization of liquid dispensing apparatus, in particular dispensers for drinks and the like.

Periodic sterilization of liquid dispensing apparatus is necessary in order to avoid a build-up of harmful organisms, which may in turn contaminate the liquid which is dispensed. Regular sterilization of fixed plant in a mass production facility can be part of a routine maintenance schedule. Sterilization in a bar or kitchen can be scheduled, but may occasionally be missed. Good hygiene is most difficult to ensure in a multi-user dispenser, such as a drinks machine, where drinks may be dispensed relatively infrequently, and a cleaning routine is dependant upon timely and effective action by a visiting machine attendant.

In most cases, existing cleaning and sterilization routines rely upon an operative washing, flushing and wiping machine components. These routines can increase or spread contamination, if correct procedures are not followed, and unfortunately it is not uncommon for such cleaning to be imperfect.

What is required is an automatic sterilization routine that can operate periodically without human intervention, and is suitable for a wide variety of liquid dispensing apparatus.

According to the present invention there is provided a liquid dispensing apparatus comprising a liquid supply duct from a source to a dispensing outlet, a liquid return duct from said outlet to a junction at the downstream side of said source, said supply duct and return duct together comprising a fluid circuit, a supply valve in the supply duct between said source and junction, a return valve in the return duct upstream of said junction, an ozone generator in said return duct, a pump in said fluid circuit, said pump being operable to circulate liquid via said ozone generator.

Preferably control means are provided and operable on demand to close said supply valve and open said return valve.

Ozone is a well-known sterilizing agent, and in a cleaning cycle is introduced into the return duct during circulation of liquid therethrough. Such circulation and re-circulation can continue for a pre-determined period until sterilization is assured. The period of sterilization can be determined empirically according to the particular dispensing apparatus, by routine methods of inspection and testing.

The ozone generator may be activated by flow of liquid in said return duct or enabled by said control means when a sterilization routine is demanded.

In a preferred embodiment a single pump is provided in the supply duct downstream of the junction so as to function in both dispensing of liquid from the dispensing outlet and in circulating around the fluid circuit. In the alternative, separate pumps may be provided, and in this case a circulating pump may be provided in the return duct. Preferably the ozone generator is upstream of the return valve.

Circulation of sterilizing fluid through the dispensing outlet ensures that all surfaces which are wetted during routine dispensing are also wetted during sterilization. In one embodiment the dispensing outlet is movable from a dispense position to a cleaning position whereby liquid running from the outlet is directed into the inlet of the return duct. Alternatively a return duct inlet may move on demand into a cleaning position in the flow path from the dispensing outlet. Either cleaning position may constitute a rest position for the dispensing outlet.

In many drinks dispensers, a chiller is provided in the supply duct downstream of the supply valve. In a preferred embodiment the chiller is downstream of said junction, and is sterilized in a cleaning cycle. A particular advantage of chilled liquid is that the take-up of ozone is improved, and thus in a preferred embodiment the chiller is enabled during a cleaning cycle so as to accelerate sterilization.

In a further embodiment, the dispensing apparatus is adapted to periodically circulate liquid via the chiller whilst the ozone generator is not enabled. Such circulation ensures that liquid in the portion of the supply duct immediately upstream of the dispensing outlet is chilled to some extend at all times, and obviates the problem that such portion tends towards ambient temperature. Customer satisfaction is thereby improved, at least in the case of drinks dispensers.

Many drinks dispensers also include a filter in the supply duct upstream of the chiller and adapted to remove contaminants from the liquid feedstock. A typical filter medium is activated charcoal or another carbon based material. In a preferred embodiment such a filter is included in the cleaning circuit at the end of a cleaning cycle (i.e. after the ozone generator is turned off), where the carbon-based filter material rapidly removes ozone and contaminants from the recirculating liquid. This step ensures that liquid in the supply duct at the end of a cleaning cycle may be dispensed at said outlet if desired. Alternatively a volume of feedstock may be used to flush the supply duct.

The invention may be incorporated in any liquid dispensing apparatus, or provided as an aftermarket fitting. Conventional plumbing fittings are utilized, the connections being made at suitable locations in the supply duct. Typically a cleaning routine may be implemented during a quiet time, for example at night.

According to a second aspect the invention provides a method of sterilizing a liquid dispensing apparatus having a supply duct and a dispensing outlet, the method comprising:
providing a liquid return duct from a dispensing outlet to a liquid feedstock inlet, said return duct and supply duct together comprising a fluid circuit,
providing an ozone generator in said return duct,
periodically circulating liquid feedstock in said fluid circuit whilst dispersing ozone therein,
ceasing ozone generation after a pre-determined period, and
cleaning ozone from said supply duct.

In one embodiment the cleaning step comprises flushing the supply duct with liquid feedstock. Preferably the cleaning step includes passing the circulating fluid through a filter to remove ozone therefrom. Fluid is passed through the filter for a period sufficient to reduce ozone levels to an acceptable minimum concentration.

The method may further include the step of passing the circulating fluid through a cooler, thereby to promote uptake of ozone.

Other features of the invention will be apparent from the following description of a preferred embodiment illustrated by way of example only in the accompanying drawings in which:

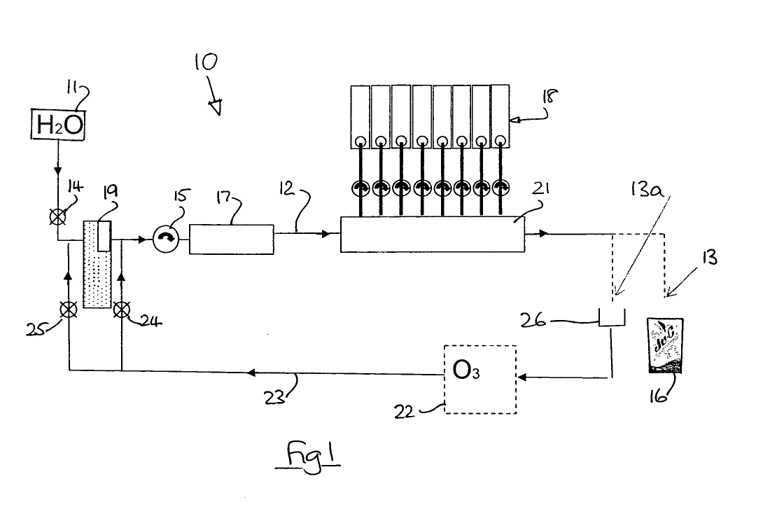

FIG. 1 is a schematic circuit diagram of a dispensing apparatus incorporating the invention.

With reference to FIG. 1, a drinks vending machine 10 comprises a source of potable water 11, for example from a mains supply, connected via a supply duct 12 to a dispensing outlet 13. A bi-stable supply valve 14 immediately downstream of the source 11 is operable to open and close the supply duct on demand. A pump 15 downstream of the supply valve is operable on demand to dispense a predetermined volume of water via the outlet 13 into a container 16.

Downstream of the pump 15 is a chiller 17 of conventional type, and a concentrate dispenser 18 whereby different flavours and additives can be used to make drinks according to a selection made by a machine user.

Upstream of the pump 15 is a filter 19 containing a carbon based material for removing contaminants from the feedstock passing through supply valve 14.

In normal use, a user will select a drink; the supply valve 14 will open to permit a volume of water to be advanced by the pump 15 through the chiller 17 to a mixing chamber 21, where the desired concentrate is added, and the mixed drink is then delivered to the outlet 13.

Periodic sterilization of the supply duct 12 is provided by a conventional ozone generator 22 in a return duct 23 which is connected to the supply duct by bi-stable valve 24 immediately downstream of the filter 19, and by bi-stable valve 25 immediately upstream of the filter.

The dispensing outlet 13 is movable between a filling position over the container 16, and a cleaning/resting position 139 over a return duct inlet 26, as illustrated.

In normal operation the dispensing outlet is in position over the container 16, valves 24 and 25 are closed, and valve 14 opens on demand to admit water to the supply duct 12.

In a cleaning cycle, the dispensing outlet moves to the cleaning/rest position over the return duct inlet 26, valves 14 and 25 are closed, valve 24 is opened, and water is circulated in a closed loop via the chiller 17 and ozone generator 22. Operation of the generator causes ozone to be diffused into the circulating liquid, gradually building-up the concentration thereof until effective sterilization is assured. The cleaning cycle runs for a pre-determined period, for example 15 minutes, which is set according to empirical testing.

The cleaning cycle is followed by a short deactivation cycle, for example 5 minutes in which valve 24 is closed and valve 25 opened. The re-circulating liquid together with any remaining contaminants is thus passed through the filter 19, which rapidly eliminates ozone and removes contaminants.

At the end of the deactivation cycle all valves close and the pump ceases ready for a regular drinks dispensing cycle.

It will be appreciated that circulation via the chiller ensures that chilled liquid is available at the dispensing outlet 13 at all times. Chilling can be ensured at times when the cleaning cycle is not required, by simply not enabling the ozone generator. Suitable temperature sensors are provided to determine temperature of liquid just upstream of the dispensing outlet, and to enable a chiller cycle accordingly. In typical use it is envisaged that plural chiller cycles will occur between each successive cleaning cycle.

The invention is suitable for all kinds of liquid dispensers, particularly drinks dispensers and most particularly vending machines. It will be appreciated however that the invention is also suitable for use in bars and restaurants, and in commercial manufacture of packaged drinks and the like.

1. A liquid dispensing apparatus comprising a liquid supply duct from a source to a dispensing outlet, a liquid return duct from said outlet to a junction at the downstream side of said source, said supply duct and return duct together comprising a fluid circuit, a supply valve in the supply duct between said source and junction, a return valve in the return duct upstream of said junction, an ozone generator in said return duct, and a pump in said fluid circuit, said pump being operable to circulate liquid via said ozone generator, wherein, in use, the supply valve is opened and the return valve closed in order to dispense liquid from the supply duct

2. Apparatus according to claim 1 and further including control means adapted on demand to close the supply valve and open the return valve to facilitate said circulation.

3. Apparatus according to claim 1 whereby said pump is provided in said supply duct downstream of said junction.

4. Apparatus according to claim 1 wherein said ozone generator is upstream of said return valve.

5. Apparatus according to claim 1 wherein said supply duct includes a chiller between said junction and outlet.

6. Apparatus according to claim 1 and further including a liquid filter connectable on demand into said fluid circuit, said filter being adapted to remove ozone from liquid flowing therethrough.

7. Apparatus according to claim 6 wherein said filter is in said supply duct.

8. Apparatus according to claim 7 wherein said filter is upstream of said junction, a filtration by-pass duct being connected between said return duct upstream of said return valve and said supply duct upstream of said filter, and a by-pass valve being provided in said by-pass duct.

9. Apparatus according to claim 8 wherein said by-pass valve and return valve are bistable.

10. A method of sterilizing a liquid dispensing apparatus having a supply duct and a dispensing outlet, the method comprising: providing a liquid return duct from said outlet to a liquid feedstock inlet, said supply duct and return duct together comprising a fluid circuit, providing an ozone generator in said return duct, circulating liquid feedstock in said fluid circuit whilst dispersing ozone therein, ceasing ozone generation after a pre-determined period, and cleaning ozone from said supply duct.

11. The method of claim 10 comprising passing circulating fluid through a filter to remove ozone therefrom after ceasing ozone generation.

12. The method of claim 10 including the step of passing circulating fluid through a chiller.
